(19) United States
(12) Patent Application Publication
(10) Pub. No.: US 2011/0251498 A1
(43) Pub. Date: Oct. 13, 2011

(54) BLOOD PRESSURE CUFF

(75) Inventors: William John Nasella, Stoneham, MA (US); Lewis Norman Harrold, Georgetown, MA (US); David Spada, East Boston, MA (US)

(73) Assignee: ANALOGIC CORPORATION, Peabody, MA (US)

(21) Appl. No.: 13/140,685

(22) PCT Filed: Dec. 23, 2008
(86) PCT No.: PCT/US08/88130

§ 371 Date: Jun. 17, 2011

(51) Int. Cl. A61B 5/022

(52) U.S. Cl. 600/490

(57) ABSTRACT

A physiological parameter determining device (100, 1000) includes a flexible band (102) configured to be releasably affixed around an extremity of a subject, a plenum (104) disposed in the flexible band (102), wherein the plenum (104) includes two or more flexible inflatable hollow channels (116) separated from each other by at least one non-inflatable region (120); and circuitry (106, 1020) physically integrated with the flexible band (102), wherein the circuitry (106, 1020) includes at least a transducer (216) that senses information indicative of a physiological parameter and generates a signal indicative of the physiological parameter.

### TECHNICAL FIELD

The following generally relates to a blood pressure cuff, and finds particular application to a cuff with a channeled plenum and integrated sensing componentry.

### BACKGROUND

Vital signs are physiological statistics that can be used to assess various bodily functions. An example of a vital sign is blood pressure, which is a measure indicative of the force exerted by circulating blood on the walls of blood vessels. Generally, blood pressure is represented in terms of a ratio of a systolic pressure (a peak pressure occurring near the beginning of the cardiac cycle) over a diastolic pressure (a minimum pressure occurring near the end of the cardiac cycle), in units of millimeters of mercury (mmHg).

Blood pressure can be measured non-invasively or invasively. Non-invasive techniques include palpation, auscultatory, and oscillometric techniques. Oscillometric blood pressure devices (commonly referred to as non-invasive blood pressure or NiBP monitors) generally include a cuff with an inflatable bladder and a pressure sensor. The bladder is connected to an electronically controlled air pump via an air hose, and the pressure sensor is electrically connected to measurement and readout circuitry via a cable. The air pump and measurement and readout circuitry are often part of a separate wall-mounted or bed-side blood pressure monitor, which can be used interchangeably with different cuffs.

For measuring blood pressure, the cuff is placed around the upper arm of a patient at about the same vertical height as the heart with the pressure sensor selectively located to sense blood flow in the upper arm. The bladder is then inflated to temporarily occlude blood flow in the upper arm. A valve is then opened to slowly deflate the bladder. As bladder pressure decreases and blood starts to flow, the transducer senses the flow and output a signal which is indicative of systolic pressure. When blood flow is no longer sensed, the output signal of the transducer is indicative of diastolic pressure. The blood pressure monitor derives the blood pressure value from the transducer output signal and displays the value.

Unfortunately, the air pumps that have been used with NiBP monitors are relatively large and heavy, and typically have to run for ten (10) to fifteen (15) seconds to inflate a standard size adult NiBP Cuff. For example, the unrestricted flow rate of an example air pump is about eighteen hundred (1800) cubic centimeters (cc)/minute, and the volume of a standard adult cuff is approximately three hundred and twenty-five (325) cc. Smaller air pumps such as five hundred (500) to eight hundred (800) cc/minutes air pumps require even more time to inflate the cuff, while larger pumps are relatively more electrically inefficient and subject to increased work loads and thus wear.

### SUMMARY

Aspects of the application address the above matters, and others. In one aspect, a physiological parameter determining device includes a flexible band configured to be releasably affixed around an extremity of a subject, a plenum disposed in the flexible band, and circuitry physically integrated with the flexible band. The plenum includes two or more flexible inflatable hollow channels that are separated from each other by at least one non-inflatable region, and the circuitry includes at least a transducer that senses information indicative of a physiological parameter and generates a signal indicative of the physiological parameter.

In another aspect, a method includes wirelessly activating circuitry of a blood pressure cuff and wirelessly conveying a signal from the blood pressure cuff. The circuitry, when activated, being configured to expand a plurality of flexible inflatable hollow channels of the cuff and occlude blood flow in an extremity of a subject, to controllably collapse the expanded flexible inflatable hollow channels of the cuff after blood flow is occluded in the extremity, to sense blood flow in the extremity as the expanded flexible inflatable hollow channels collapses, and to generate a signal indicative of the blood pressure.

In another aspect, a method includes securing a blood pressure cuff around an upper arm of a subject and activating the circuitry of the cuff. The cuff includes circuitry, a transducer, and a plenum comprised of both a plurality of interconnected flexible inflatable hollow channels and non-inflatable regions dispersed amongst the channels. The circuitry is activated to expand the channels, and thereby occlude blood flow in the upper arm, and to sense information indicative of blood pressure via the transducer.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 illustrates an example physiological parameter determining device;

FIG. 2 illustrates an example control circuitry of the physiological parameter determining device;

FIGS. 3-8 illustrate various plenum configurations;

FIG. 9 illustrates a cuff guide in connection with the physiological parameter determining device;

FIG. 10 illustrates an embodiment in which the physiological parameter determining device includes a semi-rigid side; and

FIG. 11 illustrates an example method.

### DETAILED DESCRIPTION

FIG. 1 illustrates an example physiological parameter determining device 100 that obtains information used to determine or derive a physiological parameter such a blood pressure. The illustrated device 100 includes a flexible band or cuff 102, a fluid inflatable plenum 104, and circuitry 106.

The illustrated cuff 102 is configured to be wrapped around and encircle a periphery of an extremity, such as an upper portion of an arm, a leg, and/or other extremity of a subject. The cuff 102 is generally selectively positionable on the extremity and is positioned with respect to a blood vessel, such as an artery, to be used for a blood pressure measurement.

The cuff 102 includes complementary securing regions 108 and 110, respectively located on opposing major sides of the cuff 102 such as a first outer side 112 and a second inner side 114, which faces the extremity when performing a blood pressure measurement. The regions 108, 110 are configured to matingly engage and secure the cuff 102 for a blood pressure measurement. The securing regions 110 and 108 respectively may include hook and loop type fasteners and/or other complementary fasteners.

In the illustrated embodiment, the plenum 104 is located or integrated within the cuff 102 and includes a plurality of flexible inflatable hollow channels 116, which extend along a long axis 118 of the cuff 102. Portions of the channels 116 are partially separated from each other by non-inflatable regions 120. A semi-rigid material such as high density foam, plastic, gel and/or other material can form at least part of a non-inflatable region 120.

In one instance, using such a channel based plenum 104 with non-inflatable regions 120 dispersed as such can reduce the volume of the plenum while providing for diffuse pressure over the extremity for a blood pressure measurement, relative to a configuration in which the cuff 102 includes a bladder or the like instead of the channels 116.

The flexible inflatable hollow channels 116 are configured such that they transition to or are maintained in a collapsed or semi-collapsed state when fluid is not being supplied to or maintained in the plenum 104. Otherwise, fluid in the channels 116 tends to cause the channels 116 to radially expand.

Such expansion generally is greater in a direction extending from the major surfaces 112, 114 relative to a direction along the axis 118. As discussed in greater detail below, a guide can additionally be used to reduce or inhibit expansion of the cuff 102 in a direction away from the extremity.

Various techniques can be used to secure the plenum 104 to the cuff 102. For example, in one instance the sides 112, 114 may be secured together in one or more of the non-inflatable regions 120. In yet another instance, one or more of the channels 116 can be secured to at least one of the sides 112, 114. Other approaches are also contemplated herein. Securing of the plenum 104 can be through an adhesive, a fastener, and/or otherwise.

The illustrated flexible hollow channels 116 are interconnected and form a continuing meandering pattern in which the non-inflatable regions 120 lie substantially between at each of the channels 116. Other plenum 104 configurations are also contemplated herein, as described in greater detail below.

It is to be appreciated that the location, orientation, size, shape, and/or other feature of a channel 116 may correspond to a particular extremity, blood vessels and/or other characteristics of the subject.

The circuitry 106 is physically integrated with or part of the cuff 102 and controls fluid flow into and out of the plenum 104, senses and generates a signal indicative of blood flow in the extremity, provides the signal to one or more devices that derive a blood pressure value (systolic and/or diastolic) from the signal, and/or performs other functionality.

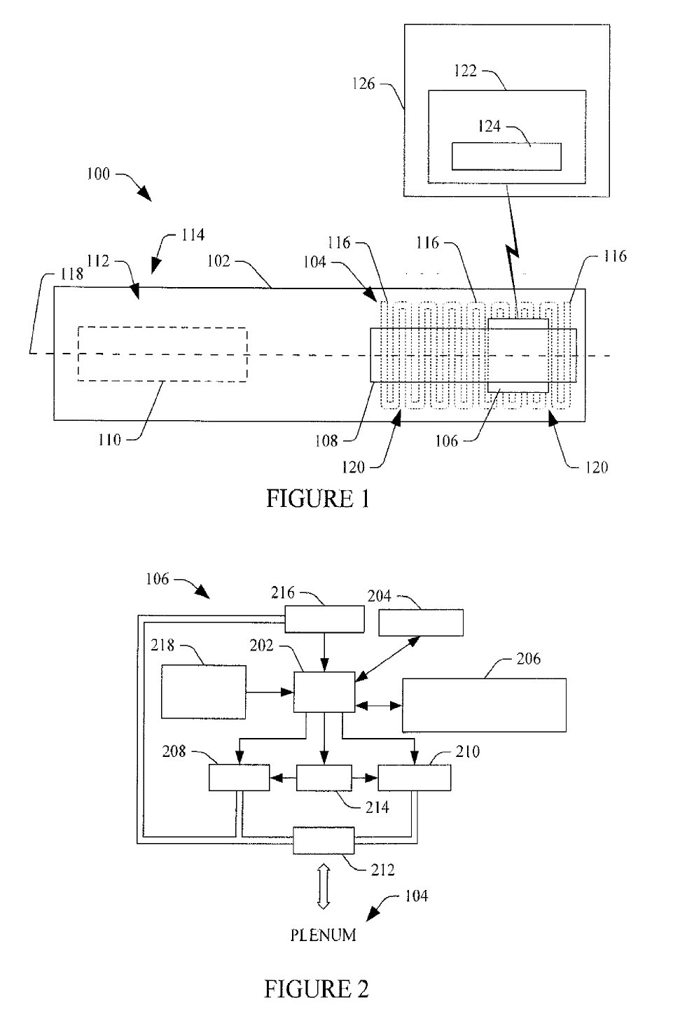

FIG. 2 illustrates example circuitry 106. As shown, in the illustrated embodiment the circuitry 106 includes a microprocessor (μCPU) 202 that controls various components of the circuitry 106.

Memory 204 includes instructions and/or algorithms that can be accessed and executed by the μCPU 202. The memory 204 and/or other memory can also be used by the μCPU 202 to perform various operations, store data, etc. In another embodiment, the memory 204 is integrated into the microprocessor 202. A communications interface 206 is configured for wireless communications such as radio frequency, optical, and/or other wireless communications. As such, the communications interface 206 can wirelessly transmits information such as a signal indicative of blood flow and/or receive information such as a signal indicative of a desired operating state (e.g., on/off). Other information such as instructions, algorithms, state, security data, patient information, and/or other information can also be transmitted and/or received. In one instance, software updates, including, but not limited to algorithm updates, may be wirelessly transmitted in the memory 204.

Transmission of the signal indicative of blood flow can be automated such that the signal is transmitted without user intervention. For example, once blood flow measurements are acquired, the circuitry 106 can automatically send them to a monitoring device. In another embodiment, the user interaction invokes such transmission. The communications interface 206 (and/or another component of the circuitry 106) may include a physical interface or port for wired communications.

A fluid pump 208 supplies fluid to the plenum 104, and a bleed or relief valve 210 releases fluid from the plenum 104. One or more ports 212 are configured to connect the fluid pump 208 and the valve 210 with the plenum 104. A controller (ctlr) 214 controls the pump 208 and the valve 210 based on a fluid pressure of the plenum 104, an instruction from the microprocessor 202, and/or otherwise.

A transducer such as a pressure sensor 216 is configured to sense blood flow in a blood vessel located in the extremity surrounded by the cuff 102. The sensor 216 generates a signal indicative of the blood flow. The sensor 216 is also connected to the port 212 and can sense the pressure corresponding to the pump 208 and/or port 212.

A power source 218 supplies power for powering components of the circuitry 106.

Returning to FIG. 1, the circuitry 106 is configured to communicate with a remote device 122. The illustrated remote device 122 includes at least one control 124 for transmitting an activation signal the circuitry 106. The control 124 can be invoked via sensory inputs such as touch or sound. This same control or another control can be configured for transmitting a deactivation signal to the circuitry 106.

The illustrated remote device 122 is part of a monitor 126 such as a bed side monitor, a hand held portable monitor, a central monitoring station, and/or other monitor. The monitor 126 can include a processor and memory with one or more suitable algorithms for deriving a blood pressure measure from the signal from the circuitry 106.

The monitor 126 can also include a display for visually presenting the blood pressure value, an analyzer that analyzes the blood pressure value with respect to a predetermined blood pressure range, a notifier that provides a visual and/or audible signal such as an alarm when the blood pressure value is outside of the predetermined range, and/or one or more other components.

In another embodiment, the remote device 122 is separate and external from the monitor 126. With this embodiment, the remote device 122 can still be used to activate and/or deactivate the circuitry 106, and the monitor can still receive the signal, derive the blood pressure value, and display the value.

As noted above, the device 100 with the cuff 102 with the channels 116 and the non-inflatable regions 120 may reduce the volume of plenum used to occlude blood flow for blood pressure measurements relative to a configuration in which the cuff includes a bladder or the like without the channels 116 and the non-inflatable regions 120.

It is to be appreciated that reducing the volume as such may decrease inflate time and/or decrease pump run time. In addition, as the power rating and/or electrical current draw of the pump generally are related to the plenum volume, reducing the plenum 104 volume allows for use of a pump with a relatively lower power rating and/or operating current draw. Reducing run time and power consumption can increase pump lifetime and/or allow for a physically smaller and/or lower power battery.

Incorporating the circuitry 106 on the cuff 102 allows for mitigation of an external fluid transporting hose, external electrical wires and/or other components connecting a cuff and a separate flow control and monitoring device. Such incorporation can also increase ease of use and/or facilitate minimizing the profile of the device 100. Wireless connectivity allows for automation of conveying information.

Other embodiments are discussed.

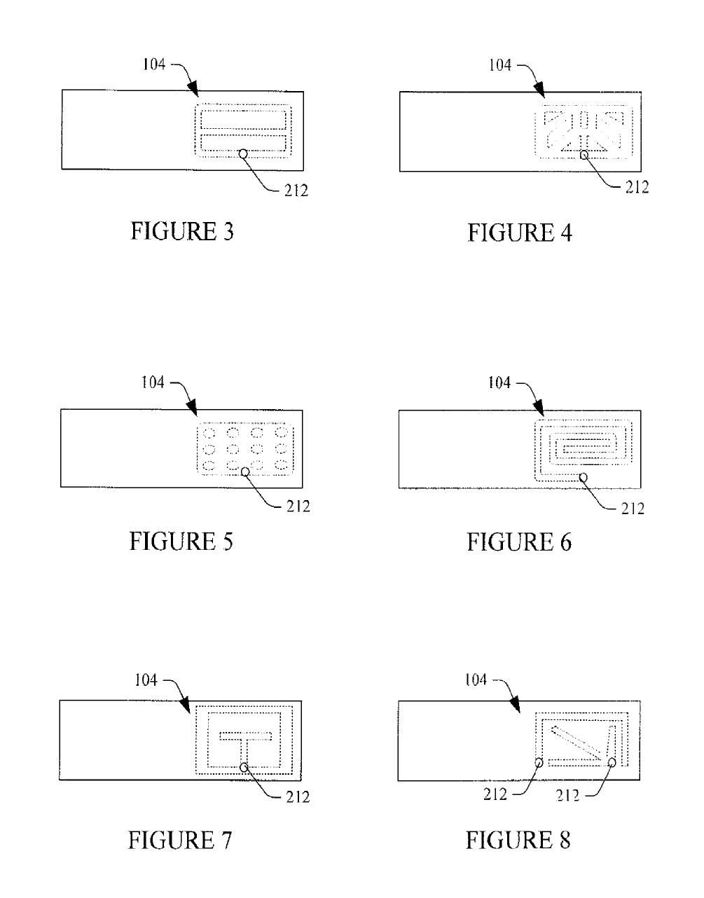

As briefly noted herein, the plenum 104 can be variously configured. FIGS. 3-8 show other non-limiting examples. In FIG. 3, the plenum 104 includes interconnected vertical and horizontal channels 116, which are supplied with fluid via the port 212. With this configuration, two or more channels extend around the radius of the arm and are connected at ends. In FIG. 4, the plenum 104 also includes diagonal channels 116.

In FIG. 5, the plenum 104 is a single chamber with a plurality of non-inflatable regions therein. In FIG. 6, the plenum 104 includes a plurality of channels interconnected to form a spiraling channel. In FIG. 7, at least two sets of channels of the plenum 104 are not interconnected. Instead, each set is independently supplied with air via the port 212. In FIG. 8, different ports 212 are used to supply fluid to different channels 116. Other configurations are also contemplated herein.

In another embodiment, the circuitry 106 executes an algorithm for deriving a blood pressure value from the signal from the sensor 216. The algorithm can be stored in the memory 204 and/or otherwise. The blood pressure value can be conveyed from the circuitry 106 via the communications interface 206. The circuitry 106 may also include a display component for displaying the derived blood pressure value.

In another embodiment, the device 100 also includes an indicator such as a visual or audible tactile indicator. In one instance, the indicator is used to indicate successful transmission of the signal. In other instances, the indicator can be used to additionally or alternatively indicate other information.

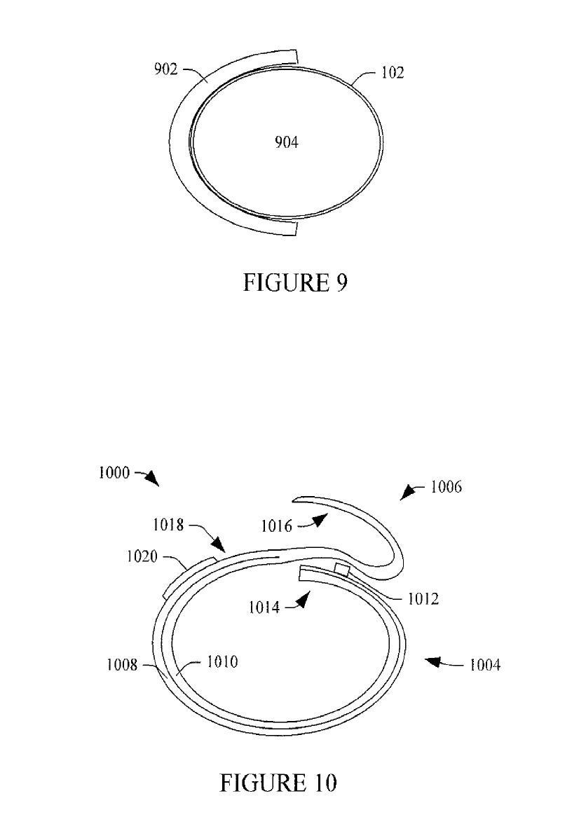

FIG. 9 shows an embodiment in which a guide 902 is employed along with the cuff 102. The guide 902 is shown installed over a sub-portion of the cuff 102, which is on an extremity 904 of a subject. In the illustrated embodiment, the guide 902 is “C” shaped and includes a rigid or semi-rigid material. In other embodiments, the guide 902 can be otherwise shaped and/or include a less rigid or more flexible material. In one instance, the guide 902, when installed over the cuff 102, inhibits expansion of the channels 116 in a direction away from the extremity, thereby, further reducing the volume of the plenum 104. The guide 902 may also facilitate securing the cuff 102 to the extremity 904. In one instance, the guide 902 is installed by slipping the guide 902 over the arm and the installed cuff 102. In another instance, the guide 902 is flexible and is manually opened, fitted over the cuff 102, and manually closed to substantially conform to the perimeter of the cuff 102.

FIG. 10 illustrates an embodiment in which a physiological parameter determining device 1000 includes a first elongate region 1004 physically attached to a second elongate region 1006. The first elongate region 1004 includes an outer semi-rigid flexible side 1008 and an inner plenum side 1010. The illustrated outer semi-rigid flexible side 1008 substantially extends along the first end region 1004. It is to be appreciated that the outer semi-rigid flexible side 1008 may extend along sixty (60) to one hundred (100) percent of the first end region 1004.

The inner plenum side 1010 is substantially similar to the cuff 102 described herein and includes a reduced volume plenum with a plurality of flexible inflatable hollow channels (not visible). A loop 1012 is located at about an end region 1014 of the first elongate region 1004. The second elongate region 1006 includes a securing region 1016 that removably engages a securing region 1018 located on the outer semi-rigid flexible side 1008. In another embodiment, the device 1000 alternatively includes securing regions substantially similar to the securing regions 108 and 110 described in connection with FIG. 1. Circuitry 1020, which is substantially similar to the circuitry 106, affixes to the outer semi-rigid flexible side 1008. In another embodiment, the circuitry 1020 is embedded in the outer semi-rigid flexible side 1008.

Generally, the first elongate region 1004 is wrapped around the extremity when using the device 1000 to obtain a blood pressure measurement. The second elongate region 1006 loops through the loop 1012 and the securing regions 1016 and 1018 are engaged. The circuitry 1020 is then activated to obtain a blood pressure measurement. The outer semi-rigid flexible side 1008 inhibits expansion of the plenum in a direction away from the extremity, reducing the volume of the plenum needed for a blood pressure measurement.

In another embodiment, the control circuitry 106 is removable from the device 100. With this embodiment, an external hose and cable can be used to connect the cuff 102 and circuitry 106. In addition, the cuff 102 can alternatively be connected to other control circuitry.

In another embodiment, one or more other sensors may be incorporated in and/or included with the cuff 102. For example, a temperature sensor can be added to the cuff 102 to provide a signal indicative of a temperature of the surface of the extremity.

In another embodiment, the plenum 104 is located externally on the cuff 102

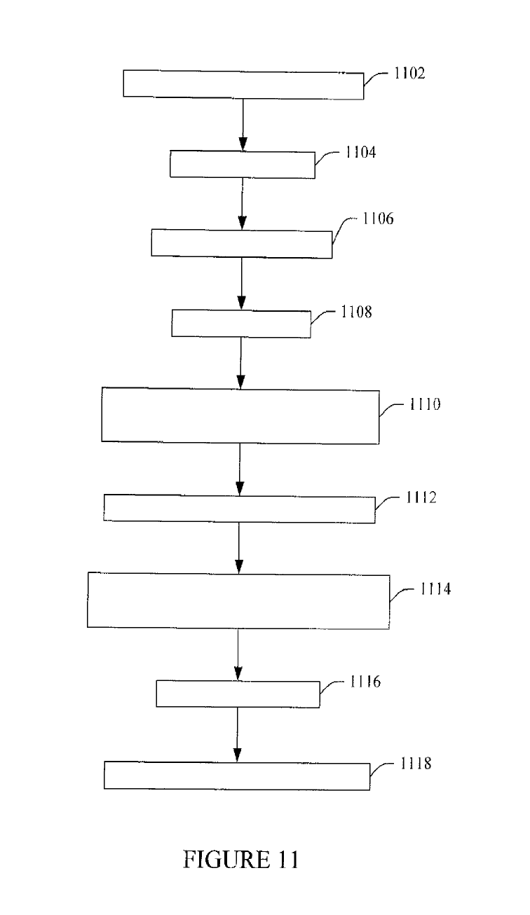

FIG. 11 illustrates a method of performing a blood pressure measurement.

At 1102, the cuff 102 is placed around and secured to the upper arm of a patient.

At 1104, the circuitry 106 and hence the cuff 102 is invoked or activated to perform a blood pressure measurement.

At 1106, the relief valve 210 is closed, if it is not already closed.

At 1108, the pump 208 is turned on and supplies fluid to the plenum 104.

At 1110, once a predetermined maximum threshold pressure is reached, the pump 208 is turned off.

At 1112, the valve 210 is controllably opened to slowly deflate the plenum 104.

At 1114, the sensor 216 senses blood flow in a blood vessel and generates a signal indicative thereof.

At 1116, the signal is sent to the remote device 122. As discussed herein, the remote device 122 may derive a blood pressure value therefrom.

At 1118, once a predetermined minimum threshold pressure is reached, the valve 210 is further opened to more quickly release remaining fluid in the plenum 104.

The control circuitry 106 can be configured to enter an idle or other state until invoked to perform another blood pressure measurement. The blood pressure device 100 may be left on the patient's arm or removed therefrom between measurements

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A physiological parameter determining device, comprising:
a flexible band configured to be releasably affixed around an extremity of a subject;
a plenum disposed in the flexible band, wherein the plenum includes two or more flexible inflatable hollow channels separated from each other by at least one non-inflatable region; and
circuitry physically integrated with the flexible band, wherein the circuitry includes at least a transducer that senses information indicative of a physiological parameter and generates a signal indicative of the physiological parameter.

2. The device of claim 1, wherein the one or more of the channels extend along a long axis of the flexible band so as to surround a perimeter of the extremity when the flexible band is releasably affixed to the extremity.

3. The device of claim 1, further including a communications port configured to wirelessly convey the signal to a remote device.

4. The device of claim 3, wherein the remote device is one of a bed side monitor, a hand held portable monitor, or a central monitoring station.

5. The device of claim 3, wherein the remote device is a remote control.

6. The device of claim 3, wherein the communications port is configured to receive an activation signal that invokes the device to sense the information and generate the signal.

7. The device of claim 1, wherein the at least one non-inflatable region includes a semi-rigid material.

8. The device of claim 1, wherein the channels maintain a collapsed or semi-collapsed state when fluid is not being supplied to or maintained in the plenum.

9. The device of claim 1, wherein the channels transition to a generally expanded state when fluid is supplied to the plenum.

10. The device of claim 1, wherein the channels maintain a generally expanded state when fluid is maintained in the plenum.

11. The device of claim 10, wherein the band occludes blood flow in a blood vessel of the extremity when the channels are in the generally expanded state.

12. The device of claim 1, wherein the channels expand in a direction towards the extremity to a greater degree relative to a direction not towards the extremity.

13. The device of claim 1, further including a guide that inhibits expansion of the plenum in a direction away from the extremity.

14. The device of claim 1, the circuitry, including:
a fluid pump that supplies fluid to the plenum based at least on a pressure of the plenum and an activation signal.

15. The device of claim 1, the circuitry, including:
a relief valve that controllably releases fluid from the plenum.

16. The device of claim 1, the circuitry, including:
a microprocessor that derives a blood pressure value based on the signal.

17. The device of claim 1, further including at least one indicator that provides at least one of a visual or an audible indication that the signal has been conveyed from the device.

18. The device of claim 1, wherein the flexible band includes a first semi-rigid side and a second side that includes the plenum.

19. The device of claim 18, wherein the circuitry is embedded in the first semi-rigid side.

20. A method, comprising:
wirelessly activating circuitry of a device, the circuitry, when activated, being configured to expand a plurality of flexible inflatable hollow channels of a cuff of the device and occlude blood flow in an extremity of a subject; controllably collapse the expanded flexible inflatable hollow channels of the cuff after blood flow is occluded in the extremity; sense blood flow in the extremity as the expanded flexible inflatable hollow channels collapses; and generate a signal indicative of the blood flow; and
wirelessly conveying the signal from the blood pressure cuff.

21. The method of claim 20, wherein at least two of the flexible inflatable hollow channels are separated by a non-inflatable region.

22. The method of claim 20, wherein the circuitry is wirelessly activated by either a remote control or a monitoring device.

23. The method of claim 20, wherein the signal is wirelessly conveyed to at least one of a bedside monitor, a hand held portable monitor or a central monitoring station.

24. A method, comprising:
securing a blood pressure cuff around an upper arm of a subject, wherein the cuff includes circuitry, a transducer, and a plenum comprised of both a plurality of interconnected flexible inflatable hollow channels and non-inflatable regions dispersed amongst the channels, and
activating the circuitry of the cuff to expand the channels, and thereby occlude blood flow in the upper arm, and to sense information indicative of blood pressure via the transducer.

25. The method of claim 24, further including wirelessly obtaining the information from the circuitry.

26. The method of claim 24, further including activating the circuitry via a remote control.
